(19) United States
(12) Patent Application Publication
(10) Pub. No.: US 2015/0045602 A1
(43) Pub. Date: Feb. 12, 2015

(54) PROCESS FOR PROMOTING DISPROPORTIONATION REACTIONS AND RING OPENING REACTIONS WITHIN AN ISOMERIZATION ZONE

(71) Applicant: UOP LLC, Des Plaines, IL (US)

(72) Inventors: Mark P. Lapinski, Aurora, IL (US); Gregory Funk, Carol Stream, IL (US)

(21) Appl. No.: 14/446,591

(22) Filed: Jul. 30, 2014

Related U.S. Application Data
(60) Provisional application No. 61/994,583, filed on May 16, 2014.
(60) Provisional application No. 61/987,348, filed on May 1, 2014.
(60) Provisional application No. 61/863,019, filed on Aug. 7, 2013.

(51) Int. Cl. C07C 5/22; C07C 6/10

(52) U.S. Cl. CPC C07C 5/2206; C07C 6/10
USPC 585/310

(57) ABSTRACT

A process for increasing disproportionation and ring opening reactions an isomerization zone which converts iso-paraffins to normal paraffins. In order to promote these reactions, the amount of C₆ cyclic hydrocarbons entering the isomerization zone is reduced. Disproportionation reaction selectivity is observed which produces valuable C₃ hydrocarbons and C₄ hydrocarbons. Also, a higher ring opening conversion of C₅ cyclic hydrocarbons is observed. Conversion of iC₄ hydrocarbons, iC₅ hydrocarbons, and iC₆ hydrocarbons may occur in the same isomerization zone.

### RELATED APPLICATIONS

This application claims priority to U.S. Patent Application No. 61/863,019, filed on Aug. 7, 2013, to U.S. Patent Application No. 61/987,348, filed on May 1, 2014, and to U.S. Patent Application No. 61/994,583, filed on May 16, 2014, all of which are incorporated herein by reference.

### FIELD OF THE INVENTION

This invention relates to processes for separating out various fractions of a naphtha stream, and more particularly to isomerizing portions of the naphtha stream in an isomerization zone while promoting disproportionation reactions and ring opening reactions.

### BACKGROUND OF THE INVENTION

Ethylene and propylene are important chemicals for use in the production of other useful materials, such as polyethylene and polypropylene. Polyethylene and polypropylene are two of the most common plastics found in use today and have a wide variety of uses as, for example, a material for fabrication or a material for packaging. Other uses for ethylene and propylene include the production of vinyl chloride, ethylene oxide, ethylbenzene and alcohol.

The great bulk of the ethylene consumed in the production of the plastics and petrochemicals such as polyethylene is produced by the thermal cracking of higher molecular weight hydrocarbons. Steam is usually mixed with a feed stream to a cracking reactor to reduce the hydrocarbon partial pressure and enhance the olefin yield and reduce the formation and deposition of carbonaceous material in the cracking reactors. The process is therefore often referred to a steam cracking or pyrolysis.

The composition of the feed stream to the steam cracking reactor affects the results. A fundamental basis of this is the propensity of some hydrocarbons to crack more easily than others. The normal ranking of tendency of the hydrocarbons to crack to ethylene is normally given as: normal paraffins; iso-paraffins; olefins; naphthenes; and, aromatics. Benzene and other aromatics are particularly resistant to steam cracking with only the alkyl side chains being cracked to produce the desired product. Therefore, benzene and other aromatics are undesirable as cracking feed stocks.

The feed stream to a steam cracking unit can be quite diverse and can be chosen from a variety of petroleum fractions. The feed stream to the subject process preferably has a boiling point range falling within the naphtha boiling point range or about 36° C. to 205° C. It is preferred that the feed stream does not contain appreciable amounts, e.g. more than 5 mole %, of C₁₂ hydrocarbons. A representative feed stream to the subject process is a C₅-C₁₁ fraction produced by fractional distillation of a hydrotreated petroleum fraction. Hydrotreating is desired to reduce the sulfur and nitrogen content of the feed down to acceptable levels. A second representative feed is a similar fraction comprising C₅ through C₉ hydrocarbons.

The feed to a steam cracking unit is also normally a mixture of hydrocarbons varying both by type of hydrocarbon and carbon number. This variety results in it being very difficult to separate less desirable feed components, such as naphthenes and aromatics, from the feed stream by fractional distillation. The hydrocarbons that are not the normal paraffins can be removed by solvent extraction or adsorption. These non-normal hydrocarbons can be upgraded to improve the feedstock to the steam cracking unit.

One way to upgrade these non-normal hydrocarbons is to pass the non-normal paraffins to an isomerization zone. In the isomerization zone, the non-normal paraffins are converted, in the presence of a catalyst, into normal paraffins.

Based upon current designs, conversion of iC₅ hydrocarbons and iC₆ hydrocarbons to normal paraffins in an isomerization zone is limited, by equilibrium conditions, to about 25% and 13% per pass, respectively. Based upon typical processing conditions, full conversion of the iso-paraffins entails large recycle streams, large fractionation columns, and large utility costs. The per pass conversion rates can be increased for example, by increasing the temperature of the isomerization zone, by lowering the liquid hourly space velocities (LHSV), or both, which leads to the cracking of some of paraffins to lighter C₄− hydrocarbons. The cracking reactions can lead to the production of undesired low value methane.

Furthermore, it has been observed elsewhere that, within the isomerization zone, the C₄ hydrocarbon isomerization activity is suppressed by C₅+ hydrocarbon concentrations greater than about 3.0 wt %, and C₆+ hydrocarbon concentrations greater than about 0.1 wt %, and C₇+ hydrocarbon concentrations greater than about 0.001 wt %. Thus, C₄ hydrocarbon isomerization is conducted in a separate process and is not combined with the isomerization of C₅ and C₆ light naphtha streams.

It would be desirable to have one or more processes that allow for isomerizing for at least iC₅ and iC₆ hydrocarbons to be efficiently and effectively converted to normal paraffins. It would also be desirable to have an isomerization process that allows for iC₄, iC₅, and iC₆ hydrocarbons to be efficiently and effectively converted to normal paraffins in the same isomerization zone.

### SUMMARY OF THE INVENTION

It has been discovered that disproportionation reactions and ring opening reactions to desirable products in an isomerization zone can occur while isomerizing iso-paraffins to normal paraffins in the same isomerization zone by reducing the amount of C₆ cyclic hydrocarbons in the stream passing into the isomerization zone.

It has been also been discovered that iC₄, iC₅, and iC₆ hydrocarbons can be processed together under conditions for isomerization with reduced suppression of the C₄ hydrocarbon isomerization activity by removing C₆+ cyclic hydrocarbons from the C₄ to C₆ feed stream resulting in higher C₄ conversions per pass.

Additional embodiments and details of the present invention are set forth in the following detailed description of the invention.

### DETAILED DESCRIPTION OF THE DRAWINGS

The drawings are simplified process diagrams in which:

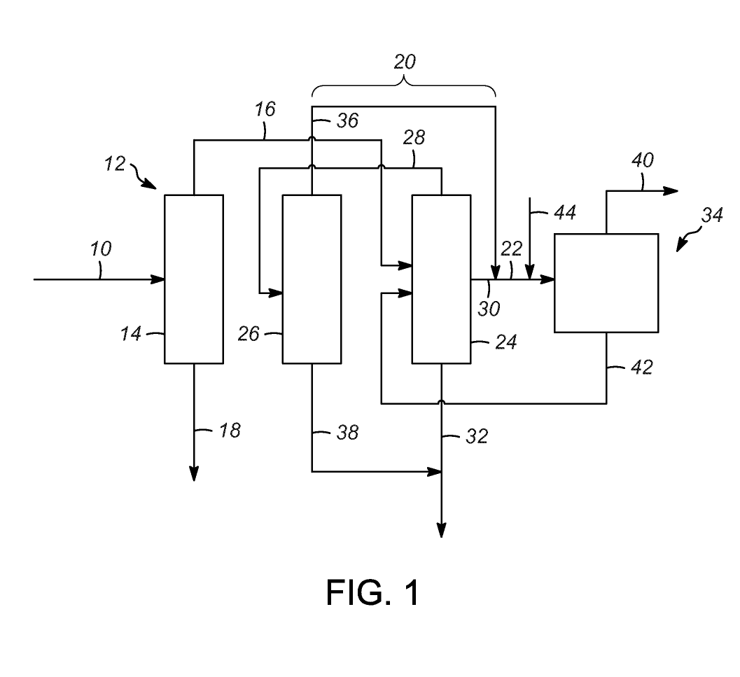

FIG. 1 shows a process flow diagram of a process according to one embodiment of the present invention;

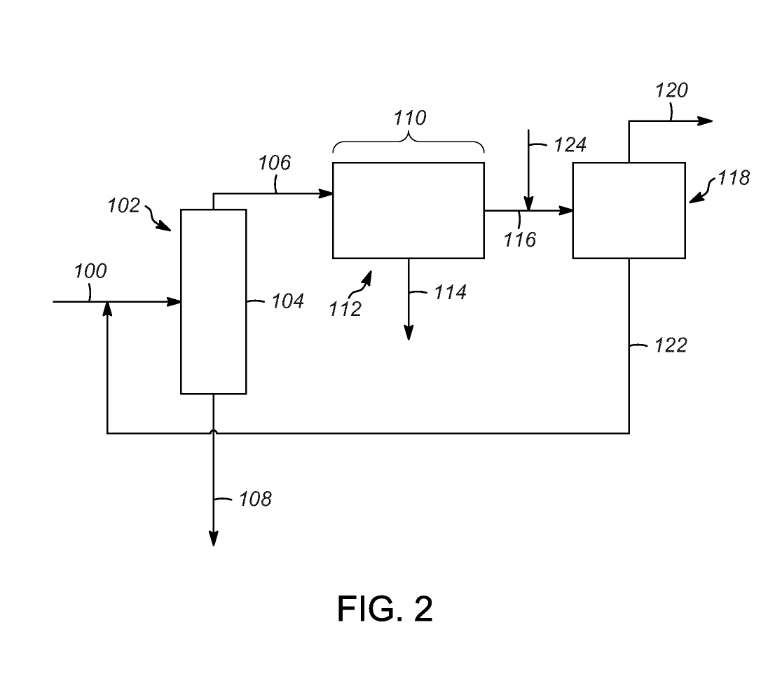

FIG. 2 shows a process flow diagram of a process according to another embodiment of the present invention; and,

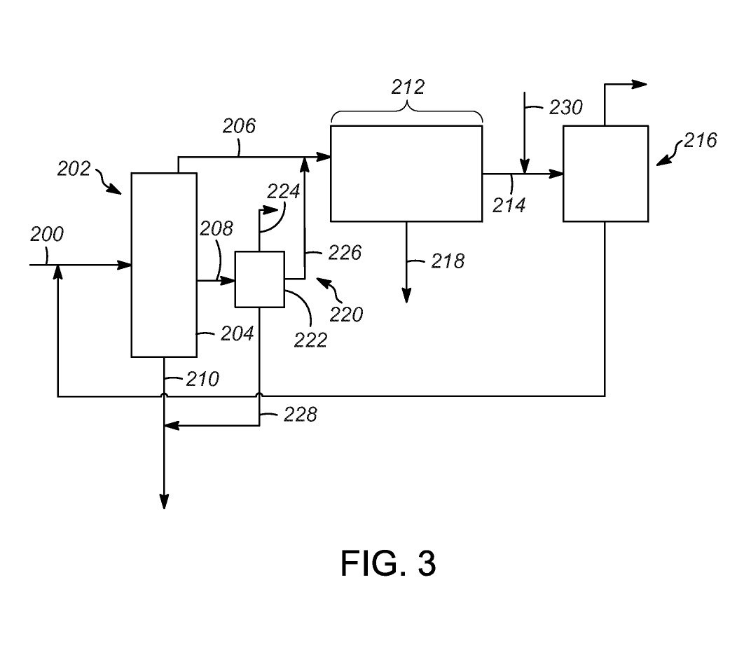

FIG. 3 shows a process flow diagram of a process according to another embodiment of the present invention.

### DETAILED DESCRIPTION OF THE INVENTION

It has been discovered that the normal paraffin yields in an isomerization zone can be increased by removing the C₆ cyclic hydrocarbons, such as cyclohexane, methyl-cyclopentane, and benzene, from the stream(s) passing into the isomerization zone. By removing the C₆ cyclic hydrocarbons, it was discovered that C₃ and normal C₄ hydrocarbons are produced via disproportionation reactions followed by C₄ isomerization reactions.

Additionally, it was discovered that by adjusting the amount of the C₆ cyclic hydrocarbons, such as cyclohexane, methyl-cyclopentane, and benzene, in the stream(s) passing into the isomerization zone allows for the yield of the isomerization zone to be selectively controlled. More specifically, by adjusting, for example, the zones used to separate the iso-paraffins from the C₆ cyclic hydrocarbons, or adding in a C₆ cyclic hydrocarbons rich stream into the isomerization zone, the rates of the disproportionation reactions and the ring opening reactions will be changed.

The ability to control the yield will allow for the creation of an optimum feed composition depending on the desired downstream processing of the various streams. In order to maximize the value with changing feed types and/or economic values, a method to regulate the formation of C₃, nC₄, normal pentane and normal hexane components in the isomerization zone would be advantageous. In addition, since disproportionation overall is exothermic, regulating the disproportionation reactions would add another method to regulate the temperature rise in the isomerization zones. The various embodiments of the present invention provide these benefits.

As mentioned above, it has been discovered that the efficient conversion of iC₄, iC₅, and iC₆ hydrocarbons to normal paraffins in the isomerization zone can be achieved by removing at least a portion of the C₆ cyclic hydrocarbons, such as cyclohexane, methyl-cyclopentane, and benzene, from the stream passed into the isomerization zone.

The ability to combine the isomerization of the C₄, C₅, and C₆ hydrocarbons will lead to lower capital expenditures and lower operating expenses because two separate isomerization zones can be combined. Thus, such a process allows for less equipment, as well as more efficient use of the remaining equipment.

Referring to the drawings, a first embodiment of the present invention is shown in FIG. 1, in which a feed stream 10 is passed into a first separation zone 12. The feed stream 10 is preferably hydrotreated naphtha comprising C₅+ hydrocarbons (meaning hydrocarbons having five or more carbon atoms).

The first separation zone 12 may include a column 14, such as a fractionation column. As will be appreciated, the depiction of column 14 is simplified as all the auxiliary operational components, such as controls, trays, condenser and reboiler, may be of conventional design. The feed stream 10, or multiple feed streams, can be fed into the column 14 at different locations if appropriate. The column 14 will typically contain conventional vapor-liquid contacting equipment such as trays or packing. The type of tray and design details such as tray type, tray spacing and layout may vary within the column 14.

The column 14 will separate the feed stream 10 into an overhead stream 16 and a bottoms stream 18. The overhead stream 16 may comprise C₅ hydrocarbons and iC₆ hydrocarbons. Since at least a portion of the C₆ cyclic hydrocarbons have been removed from the portion of the feed stream 10 in the overhead stream 16, the overhead stream 16 will be a C₆ cyclic hydrocarbons lean stream. The bottoms stream 18 may comprise n-hexane, C₆ cyclic hydrocarbons, and C₇+ hydrocarbons. Furthermore, depending on the operating conditions of the column 14, the bottoms stream 18 may also contain some small amounts of iC₆ hydrocarbons, such as 3-methylpentane.

The bottoms stream 18 may be passed to various other zones, such as, for example: to saturation and then to a steam cracker; to a reformer and then to an aromatic complex; to saturation, then to a ring operating reactor, and then to a steam cracker; or a combination of the foregoing. The further processing of the bottoms stream 18 is not necessary for the understanding and practicing of the present invention.

Returning to FIG. 1, the overhead stream 16 from the first separation zone 12 may be passed to a second separation zone 20. The second separation zone 20 provides at least one stream 22 that is rich in iC₅ hydrocarbons, iC₆ hydrocarbons, or both. In a preferred embodiment, the second separation zone 20 comprises at least two columns 24, 26. These two columns 24, 26 may also be fractionation columns.

A first column 24 in the second separation zone 20 may receive the overhead stream 16 from the first separation zone 12. In this embodiment of the present invention, the first column 24 separates the overhead stream 16 into three streams, and thus may comprise a divided wall column. Such divided wall columns are known, for example, from U.S. Pat. No. 6,927,314, the entirety of which is incorporated herein by reference. The three streams produced by the first column 24 are an overhead stream 28, an intermediate stream 30, and a bottoms stream 32.

The overhead stream 28 comprises C₅ hydrocarbons. The intermediate stream 30 comprises iC₆ hydrocarbons. The bottoms stream 32 comprises C₆ cyclic hydrocarbons and C₇+ hydrocarbons and n-hexane which either were not separated out in the first separation zone 12 or which were formed in the isomerization zone (discussed below).

The bottoms stream 32 may be passed to various other zones, such as, for example a steam cracker. The further processing of the bottoms stream 32 is not necessary for the understanding and practicing of the present invention.

The intermediate stream 30 has a high concentration of iC₆ hydrocarbons, compared to the concentration of iC₆ hydrocarbons in the feed stream 10. Thus, the intermediate stream 30 is considered an iC₆ hydrocarbon rich stream. The intermediate stream 30 may be passed to an isomerization zone 34, discussed in more detail below.

The overhead stream 28 from the first column 24 is passed to a second column 26 in the second separation zone 20. In the second column 26, the overhead stream 28 from the first column 24 of the second separation zone 20 is separated into an overhead stream 36 and a bottoms stream 38. The bottoms stream 38 comprises n-pentane and may be combined with bottoms stream 32 from the first column 24 in the second separation zone 20 and passed to, for example, a steam cracker. The further processing of the bottom stream 38 is not necessary for the understanding and practicing of the present invention.

The overhead stream 36 from the second column 26 in the second separation zone 20 comprises iC₅ hydrocarbons. Since the concentration of iC₅ hydrocarbons in the overhead stream 36 is higher than the concentration of iC₅ hydrocarbons in the feed stream 10, it is an iC₅ hydrocarbon rich stream. The overhead stream 36 from the second column 26 of the second separation zone 20 may be passed to the isomerization zone 34, discussed below. The overhead stream 36 may be combined with the intermediate stream 30 from the first column 24 of the second separation zone 20. Since both the iC₅ hydrocarbons and the iC₆ hydrocarbons streams 36, 30 were separated from at least a portion of a C₆ cyclic hydrocarbons lean stream (i.e., overhead stream 16), the amount of C₆ cyclic hydrocarbons passed to the isomerization zone 34 is lower.

In the isomerization zone 34, iC₅ hydrocarbons and iC₆ hydrocarbons, in the presence of hydrogen and a catalyst, are converted into normal paraffins. The isomerization zone 34, as is known, typically contains a series of reactors and a separation column. It is preferred that both the iC₅ hydrocarbons and the iC₆ hydrocarbons streams 36, 30 are passed to the same isomerization zone 34; however it is contemplated to utilize two separate isomerization zones.

While it is known that cracking of some of the paraffins can occur in an isomerization zone 34 to form C₄− hydrocarbons, the conversion of iC₅ and iC₆ hydrocarbons increases significantly via disproportionation reactions due to the fact that the stream(s) passed into the isomerization zone 34 are lean in C₆ cyclic hydrocarbons. It is believed that the disproportionation reactions occur by the combination of two iso-paraffin hydrocarbons followed by scission into one lighter hydrocarbon and one heavier hydrocarbon. For example, two iC₅ hydrocarbons can combine and form an iC₄ hydrocarbon and an iC₆ hydrocarbon in the presence of hydrogen. The iC₄ hydrocarbon can further react via disproportionation to form a C₃ hydrocarbon and an iC₅ hydrocarbon. A significant portion of the produced iC₄ hydrocarbons also converts to nC₄ hydrocarbons via isomerization reactions in the isomerization zone. A surprising result of the present invention is the production of C₃ and C₄ and C₇ normal paraffins via disproportionation and isomerization reactions with low production of low-value undesired methane as a cracked product. Thus, with the products of the disproportionation and isomerization reactions, there is an increase in the overall yield of the normal paraffins.

This surprising result is enabled by the use of an isomerization catalyst such as chlorided alumina, sulfated zirconia, tungstated zirconia or zeolite-containing isomerization catalysts. The isomerization catalyst may be amorphous, e.g. based upon amorphous alumina, or zeolitic. A zeolitic catalyst would still normally contain an amorphous binder. The catalyst may comprise a sulfated zirconia and platinum as described in U.S. Pat. No. 5,036,035 and European patent application 0 666 109 A1 or a platinum group metal on chlorided alumina as described in U.S. Pat. No. 5,705,730 and U.S. Pat. No. 6,214,764. Another suitable catalyst is described in U.S. Pat. No. 5,922,639. U.S. Pat. No. 6,818,589 discloses a catalyst comprising a tungstated support of an oxide or hydroxide of a Group IVB (IUPAC 4) metal, preferably zirconium oxide or hydroxide, at least a first component which is a lanthanide element and/or yttrium component, and at least a second component being a platinum-group metal component. These documents are incorporated herein for their teaching as to catalyst compositions, isomerization operating conditions and techniques.

Contacting within the isomerization zone 34 may be effected using the catalyst in a fixed-bed system, a moving-bed system, a fluidized-bed system, or in a batch-type operation. The reactants may be contacted with the bed of catalyst particles in upward, downward, or radial-flow fashion. The reactants may be in the liquid phase, a mixed liquid-vapor phase, or a vapor phase when contacted with the catalyst particles, with a mixed phase or vapor phase being preferred. The isomerization zone 34 may be in a single reactor or in two or more separate reactors with suitable means there between to insure that the desired isomerization temperature is maintained at the entrance to each zone. Two or more reactors in sequence enable improved isomerization through control of individual reactor temperatures and for partial catalyst replacement without a process shutdown.

Isomerization conditions in the isomerization zone 34 include reactor temperatures usually ranging from about 40° C. to 250° C. Reactor operating pressures generally range from about 100 kPa to 10 MPa absolute, preferably between about 0.5 and 4 MPa absolute. Liquid hourly space velocities range from about 0.2 to about 25 volumes of isomerizable hydrocarbon feed per hour per volume of catalyst, with a range of about 0.5 to 15 h⁻¹ being preferred.

Hydrogen is admixed with or remains with the isomerization feed to the isomerization zone to provide a mole ratio of hydrogen to hydrocarbon feed of from about 0.01 to 20. The hydrogen may be supplied totally from outside the process or supplemented by hydrogen recycled to the feed after separation from isomerization reactor effluent. Light hydrocarbons and small amounts of inerts such as nitrogen and argon may be present in the hydrogen. Water should be removed from hydrogen supplied from outside the process, preferably by an adsorption system as is known in the art.

Especially where a chlorided catalyst is used for isomerization, the isomerization reaction effluent can be contacted with a sorbent to remove any chloride components such as disclosed in U.S. Pat. No. 5,705,730.

Returning to FIG. 1, in order to control and adjust the disproportionation reactions and the ring opening of cyclopentane in the isomerization zone, a C₆ cyclic hydrocarbon rich stream 44 may be introduced into the isomerization zone. The C₆ cyclic hydrocarbon rich stream 44 may be adjustably controlled so that the amount may be varied. Additionally, and alternatively, various operating parameters of the first separation zone 12, the second separation zone 20, or both may be adjusted so as to increase the amount of C₆ cyclic hydrocarbons passed to the isomerization zone 34. The varying amount of C₆ cyclic hydrocarbons in the isomerization zone 34 will adjust the rates of the disproportionation reactions, the ring opening reactions, and the C₄ isomerization reactions resulting in an altered product distribution.

A first stream 40 recovered from the isomerization zone comprises C₄− hydrocarbons. The first stream 40 may be sent to gas treatment and then to a steam cracker, or it may be sent to gas treatment and separation and an iC₄ hydrocarbons stream may be sent to another isomerization zone. The further processing of the first stream 40 is not necessary for the understanding and practicing of the present invention, except that since the disproportionation reactions and the ring opening of cyclopentane produce these products, the downstream processing will impact the level of C₆ cyclic hydrocarbons introduced into the isomerization zone.

A second stream 42 recovered from the isomerization zone 34 will comprise C₅+ hydrocarbons, including normal paraffins. The second stream 42 may be sent back through the first separation zone 12, the second separation zone 20, or both to separate out the normal paraffins from the iso-paraffins. For example, as shown in FIG. 1, the second stream 42 is passed back to the first column 24 of the second separation zone 20. The normal hydrocarbons in the second stream 42 will be separated out with the C₆ cyclic hydrocarbons lean stream 16 passing through the second separation zone 20 and can be further processed as mentioned above.

Turning to FIG. 2, another embodiment of the present invention is shown. In this embodiment, a feed stream 100 is also passed into a first separation zone 102. The feed stream 100 is preferably hydrotreated naphtha comprising C₅+ hydrocarbons.

The first separation zone 102 may include a separator column 104, such as a fractionation column which preferably functions identically to the column 14 in the embodiment shown in FIG. 1. Thus, the feed stream 100 will separate into an overhead stream 106 and a bottom stream 108. However, it is also contemplated that the first separation zone 102 comprises an adsorption zone (discussed below).

The overhead stream 106 may comprise C₅ hydrocarbons and iC₆ hydrocarbons similar to the overhead stream 16 in the embodiment in FIG. 1. Since the C₆ cyclic hydrocarbons have been removed from the portion of the feed stream 100 in the overhead stream 106, the overhead stream 106 will be a C₆ cyclic hydrocarbons lean stream. The bottom stream 108 may comprise n-hexane, C₆ cyclic hydrocarbons, and C₇+ hydrocarbons and a small amount of iC₆ hydrocarbons. The bottom stream 108 may be passed to various other zones, such as, for example: to saturation and then to a steam cracker; to a reformer and then to an aromatic complex; to saturation, then to a ring operating reactor, and then to a steam cracker; or a combination of the foregoing. The further processing of bottom stream 108 is not necessary for the understanding and practicing of the present invention.

Returning to FIG. 2, the overhead stream 106 from the first separation zone 102, may be passed to a second separation zone 110. In this embodiment of the present invention, it is contemplated that the second separation zone 110 comprises an adsorption zone 112.

The adsorption zone 112 can include, as is known, a single large bed of adsorbent or in several parallel beds on a swing bed basis. However, it has been found that simulated moving bed adsorptive separation provides several advantages such as high purity and recovery. Therefore, many commercial scale petrochemical separations especially for the recovery of mixed paraffins are performed using simulated countercurrent moving bed (SMB) technology. Further details on equipment and techniques for operating an SMB process may be found in U.S. Pat. Nos. 3,208,833; 3,214,247; 3,392,113; 3,455,815; 3,523,762; 3,617,504; 4,006,197; 4,133,842; and 4,434,051, all of which are incorporated by reference in their entirety. A different type of simulated moving bed operation which can be performed using similar equipment, adsorbent and conditions but which simulates co-current flow of the adsorbent and liquid in the adsorption chambers is described in U.S. Pat. Nos. 4,402,832 and 4,498,991, which are incorporated by reference in their entirety.

Operating conditions for the adsorption chamber used in the subject invention include, in general, a temperature range of from about 20° C. to about 250° C. Adsorption conditions also preferably include a pressure sufficient to maintain the process fluids in liquid phase; which may be from about atmospheric to about 4.14 MPag (about 600 psig). Desorption conditions generally include the same temperatures and pressure as used for adsorption conditions. It is generally preferred that an SMB process is operated with an A:F flow rate through the adsorption zone in the broad range of about 1:1 to 5:0.5 where A is the volume rate of “circulation” of selective pore volume and F is the feed flow rate. The practice of the subject invention requires no significant variation in operating conditions or desorbent composition within the adsorbent chambers. That is, the adsorbent preferably remains at the same temperature throughout the process during both adsorption and desorption.

The adsorbent used in the first adsorption zone preferably comprises aluminosilicate molecular sieves having relatively uniform pore diameters of about 5 angstroms. This is provided by commercially available type 5A molecular sieves produced by UOP LLC.

A second adsorbent which could be used in the adsorption zone comprises silicalite. Silicalite is well described in the literature. It is disclosed and claimed in U.S. Pat. No. 4,061,724 issued to Grose et al., which is incorporated by reference in its entirety. A more detailed description is found in the article, “Silicalite, A New Hydrophobic Crystalline Silica Molecular Sieve,” Nature, Vol. 271, Feb. 9, 1978 which is incorporated herein by reference for its description and characterization of silicalite. Silicalite is a hydrophobic crystalline silica molecular sieve having intersecting bent-orthogonal channels formed with two cross-sectional geometries, 6 Å circular and 5.1-5.7 Å elliptical on the major axis. This gives silicalite great selectivity as a size selective molecular sieve. Due to its aluminum free structure composed of silicon dioxide, silicalite does not show ion-exchange behavior. Silicalite is also described in U.S. Pat. Nos. 5,262,144; 5,276,246 and 5,292,900, which are incorporated by reference in their entirety. These basically relate to treatments which reduce the catalytic activity of silicalite to allow its use as an adsorbent.

The active component of the adsorbent is normally used in the form of particle agglomerates having high physical strength and attrition resistance. The agglomerates contain the active adsorptive material dispersed in an amorphous, inorganic matrix or binder, having channels and cavities therein which enable fluid to access the adsorptive material. Methods for forming the crystalline powders into such agglomerates include the addition of an inorganic binder, generally a clay comprising a silicon dioxide and aluminum oxide, to a high purity adsorbent powder in a wet mixture. The binder aids in forming or agglomerating the crystalline particles. The blended clay-adsorbent mixture may be extruded into cylindrical pellets or formed into beads which are subsequently calcined in order to convert the clay to an amorphous binder of considerable mechanical strength. The adsorbent may also be bound into irregular shaped particles formed by spray drying or crushing of larger masses followed by size screening. The adsorbent particles may thus be in the form of extrudates, tablets, spheres or granules having a desired particle range, preferably from about 16 to about 60 mesh (Standard U.S. Mesh) (1.9 mm to 250 microns). Clays of the kaolin type, water permeable organic polymers or silica are generally used as binders.

The active molecular sieve component of the adsorbent will preferably be in the form of small crystals present in the adsorbent particles in amounts ranging from about 75 to about 98-wt. % of the particle based on volatile-free composition. Volatile-free compositions are generally determined at 900° C., after the adsorbent has been calcined, in order to drive off all volatile matter. The remainder of the adsorbent will generally be the inorganic matrix of the binder present in intimate mixture with the small particles of the silicalite material. This matrix material may be an adjunct of the manufacturing process for the silicalite, for example, from the intentionally incomplete purification of the silicalite during its manufacture.

Those skilled in the art will appreciate that the performance of an adsorbent is often greatly influenced by a number of factors not related to its composition such as operating conditions, feed stream composition and the water content of the adsorbent. The optimum adsorbent composition and operating conditions for the process are therefore dependent upon a number of interrelated variables. One such variable is the water content of the adsorbent which is expressed herein in terms of the recognized Loss on Ignition (LOI) test. In the LOI test the volatile matter content of the zeolitic adsorbent is determined by the weight difference obtained before and after drying a sample of the adsorbent at 500° C. under an inert gas purge such as nitrogen for a period of time sufficient to achieve a constant weight. For the subject process it is preferred that the water content of the adsorbent results in an LOI at 900° C. of less than 7.0% and preferably within the range of from 0 to 4.0 wt %.

An important characteristic of an adsorbent is the rate of exchange of the desorbent for the extract component of the feed mixture materials or, in other words, the relative rate of desorption of the extract component. This characteristic relates directly to the amount of desorbent material that must be employed in the process to recover the extract component from the adsorbent. Faster rates of exchange reduce the amount of desorbent material needed to remove the extract component, and therefore, permit a reduction in the operating cost of the process. With faster rates of exchange, less desorbent material has to be pumped through the process and separated from the extract stream for reuse in the process. Exchange rates are often temperature dependent. Ideally, desorbent materials should have a selectivity equal to about 1 or slightly less than 1 with respect to all extract components so that all of the extract components can be desorbed as a class with reasonable flow rates of desorbent material, and so that extract components can later displace desorbent material in a subsequent adsorption step.

U.S. Pat. No. 4,992,618 issued to S. Kulprathipanja, and which is incorporated by reference in its entirety, describes the use of a “prepulse” of a desorbent component in an SMB process for recovering normal paraffins. The prepulse is intended to improve the recovery of the extract normal paraffins across the carbon number range of the feed. The prepulse enters the adsorbent chamber at a point before (downstream) the feed injection point. A related SMB processing technique is the use of “zone flush.” The zone flush forms a buffer zone between the feed and extract bed lines to keep the desorbent from entering the adsorption zone. While the use of a zone flush requires a more complicated, and thus more costly rotary valve, the use of zone flush is preferred in the adsorption zones when high purity extract product are desired. In practice, a quantity of the mixed component desorbent recovered overhead from the extract and raffinate columns may be passed into a separate splitter column. A high purity stream of the lower strength component of the mixed component desorbent is recovered and used as the zone flush stream. Further information on the use of dual component desorbents and on techniques to improve product purity such as the use of flush streams may be obtained from U.S. Pat. Nos. 3,201,491; 3,274,099; 3,715,409; 4,006,197 and 4,036,745 which are incorporated herein by reference in their entirety for their teaching on these aspects of SMB technology.

It has become customary in the art to group the numerous beds in the SMB adsorption chamber(s) into a number of zones. Usually the process is described in terms of 4 or 5 zones. First contact between the feed stream and the adsorbent is made in Zone I, the adsorption zone. The adsorbent or stationary phase in Zone I becomes surrounded by liquid which contains the undesired isomer(s), that is, the raffinate. This liquid is removed from the adsorbent in Zone II, referred to as a purification zone. In the purification zone the undesired raffinate components are flushed from the void volume of the adsorbent bed by a material which is easily separated from the desired component by fractional distillation. In Zone III of the adsorbent chamber(s) the desired isomer is released from the adsorbent by exposing and flushing the adsorbent with the desorbent (mobile phase). The released desired isomer and accompanying desorbent are removed from the adsorbent in the form of the extract stream. Zone IV is a portion of the adsorbent located between Zones I and III which is used to segregate Zones I and III. In Zone IV desorbent is partially removed from the adsorbent by a flowing mixture of desorbent and undesired components of the feed stream. The liquid flow through Zone IV prevents contamination of Zone III by Zone I liquid by flow cocurrent to the simulated motion of the adsorbent from Zone III toward Zone I. A more thorough explanation of simulated moving bed processes is given in the Adsorptive Separation section of the Kirk-Othmer Encyclopedia of Chemical Technology at page 563. The terms “upstream” and “downstream” are used herein in their normal sense and are interpreted based upon the overall direction in which liquid is flowing in the adsorbent chamber. That is, if liquid is generally flowing downward through a vertical adsorbent chamber, then upstream is equivalent to an upward or higher location in the chamber.

In an SMB process the several steps e.g. adsorption and desorption, are being performed simultaneously in different parts of the mass of adsorbent retained in the adsorbent chamber(s) of the process. If the process was being performed with two or more adsorbent beds in a swing bed system then the steps may be performed in a somewhat interrupted basis, but adsorption and desorption will most likely occur at the same time.

Returning to FIG. 2, a first stream 114 and a second stream 116 are recovered from the adsorption zone 112. The first stream 114 comprises normal paraffins. The first stream 114, being rich in normal paraffins, may be sent to, for example, a stream cracker.

The second stream 116 recovered from the adsorption zone 112 comprises iso-paraffins, or is rich in iC₅ and iC₆ hydrocarbons. The second stream 116 is passed to an isomerization zone 118. As with the isomerization zone 34 in the embodiment shown in FIG. 1, in the isomerization zone 118 of this embodiment, iC₅ and iC₆ hydrocarbons, in the presence of hydrogen and an isomerization catalyst, are converted into normal paraffins.

As the second stream 116 introduced into the isomerization zone 118 is lean in C₆ cyclic hydrocarbons, there is a surprising and unexpected increase in the yields to desirable normal paraffins believed to be produced via disproportionation and isomerization reactions. The specifics of this isomerization zone 118 may be the same as discussed above, and thus, are incorporated herein to the discussion of this embodiment.

In order to control the selectivity of the disproportionation reactions and the ring opening of cyclopentane, the amount of C₆ cyclic hydrocarbons in the isomerization zone 118 may be adjusted. For example, a C₆ cyclic hydrocarbon rich stream 124 may be introduced into the isomerization zone 118. The C₆ cyclic hydrocarbon rich stream 124 may be selectively controlled so that the amount may be varied. Additionally, and alternatively, various operating parameters of the first separation zone 102, the adsorption zone 112, or both may be adjusted so as to increase the amount of C₆ cyclic hydrocarbons passed to the isomerization zone 118. The varying amount of C₆ cyclic hydrocarbons in the isomerization zone 118 will adjust the rates of the disproportionation reactions, the ring opening reactions, and the C₄ isomerization reactions resulting in an altered product distribution.

At least two streams 120 and 122 may also be recovered from the isomerization zone 118. A first stream 120 comprises C₄− hydrocarbons. The first stream 120 may be sent to gas treatment and then to a steam cracker, or it may be sent to gas treatment and separation and an iC₄ hydrocarbons stream may be sent to another isomerization zone. The further processing of the first stream 120 is not necessary for the understanding and practicing of the present invention, except that the downstream processing may have an impact on the level of C₆ cyclic hydrocarbons passed to the isomerization zone 118 based upon the desired yield from same.

The second stream 122 recovered from the isomerization zone 118 will comprise C₅+ hydrocarbons, including normal paraffins. The second stream 122 may be recycled or passed back to the first separation zone 102, the second separation zone 110, or both to separate out the normal paraffins from the iso-paraffins. For example, as shown in FIG. 2, the second stream 122 may be passed back to the first separation zone 102. It may or may not be combined with fresh feed stream 100 entering the first separation zone 102. The normal hexane will be separated out in the first separation zone 102, while the normal pentane will be separated out in the second separation zone 110. The normal paraffins will be passed along to further processing units, as discussed above.

In a third embodiment according to the present invention as shown in FIG. 3, a feed stream 200 is also passed into a first separation zone 202. The feed stream 200 is preferably hydrotreated naphtha comprising C₅+ hydrocarbons.

The first separation zone 202 may also include a column 204, such as a fractionation column. This column 204 will separate the feed stream 200 into an overhead stream 206, an intermediate stream 208, and a bottom stream 210.

The overhead stream 206 may comprise C₅ hydrocarbons and iC₆ hydrocarbons. The intermediate stream 208 may comprise n-hexane and C₆ cyclic hydrocarbons. The bottom stream 210 may comprise C₇+ hydrocarbons. The bottom stream 210 may be passed to various other zones, such as, for example: to saturation and then to a steam cracker; to a reformer and then to an aromatic complex; to saturation, then to a ring operating reactor, and then to a steam cracker; or a combination of the foregoing. The further processing of the bottom stream 210 is not necessary for the understanding and practicing of the present invention.

The overhead stream 206 may be passed to a second separation zone 212. Since the C₆ cyclic hydrocarbons have been removed from the portion of the feed stream 200 in the overhead stream 206, the overhead stream 206 will be a C₆ cyclic hydrocarbons lean stream.

It is contemplated that the second separation 212 is either a plurality of separation columns (such as the second separation zone 20 in the embodiment shown in FIG. 1), or an adsorption zone (such as the second separation zone 110 shown in FIG. 2). Accordingly, the portions of those embodiments are incorporated herein.

A first stream 214 from the second separation zone 212, rich in iso-paraffins, is passed to an isomerization zone 216. A second stream 218, rich in normal paraffins, from the second separation zone 212 may be passed to further processing zones. The isomerization zone 216 and the processing of the second stream 218 from the second separation zone 212 may be the same as discussed above with respect to the other embodiments of the present invention.

Returning to the first separation zone 202 in FIG. 3, the difference between this embodiment and the previously discussed embodiments is the intermediate stream 208 which is passed to a ring opening reaction zone 220. In the ring opening reaction zone 220, the cyclic hydrocarbons, in the presence of a catalyst, are converted into straight chain hydrocarbons. Typically, such reactions occur in a ring opening reactor 222. Such ring opening reactors are known, for example, as disclosed in U.S. Pat. Pub. No. 2005/0101814, incorporated herein by reference.

The products of the ring opening reactor 222, which can include methane to C₇+ hydrocarbons, may be separated into a C₄− hydrocarbon stream 224, a C₅ hydrocarbons and C₆ hydrocarbons stream 226, and a C₆ cyclic hydrocarbons and C₇+ hydrocarbons stream 228. The C₆ cyclic hydrocarbons and C₇+ hydrocarbons stream 228 may be combined with the bottoms stream 210 from the first separation zone 202. The C₄− hydrocarbon stream 224 may be passed to further processing units or zones. The C₅ hydrocarbon and C₆ hydrocarbon stream 226 may be combined with the overhead stream 206 of the first separation zone 202, and thus passed to the second separation zone 212 and isomerization zone 216.

In order to control and adjust the yield of the isomerization zone in this embodiment of the present invention, a C₆ cyclic hydrocarbon rich stream 230 may be introduced into the isomerization zone 216. This stream may be adjustably controlled so that the amount may be varied. Additionally, and alternatively, various operating parameters of the first separation zone 202, the second separation zone 212, the ring opening reaction zone 220, or a combination of these, may be selectively controlled so as to increase the amount of C₆ cyclic hydrocarbons passed to the isomerization zone 216. Again, varying the amount of C₆ cyclic hydrocarbons in the isomerization zone 216 will adjust the rates of the disproportionation reactions, the ring opening reactions, and the C₄ isomerization reactions resulting in an altered product distribution.

The above described embodiments are merely exemplary, and it is contemplated that other schemes and processes to provide a stream that contains a varying amount of C₆ cyclic hydrocarbons to an isomerization zone may be practiced and still fall within the scope of the present invention. Additional embodiments include recycling of the C₄ hydrocarbons produced in the isomerization zone to the separation zones and inclusion of C₄ hydrocarbons in the hydrotreated naphtha feed stream.

To demonstrate the advantages of the present invention, a chlorided-alumina catalyst that contained platinum was loaded and operated under isomerization conditions of 3.1 MPa (450 psig), with a 0.06 outlet hydrogen to hydrocarbon feed (H₂/HC) mole ratio, and at a rate of 2 h⁻¹ LHSV with an average temperature of approximately 174.4° C. (346° F.).

A feed comprising 97% iC₅+3% nC₅ hydrocarbons and that contained no C₆ cyclic hydrocarbons was processed. As shown in Table 1, significant quantities of C₆ and C₄ hydrocarbons were made via disproportionation reactions, specifically 2iC₅→iC₄+iC₆. The normal paraffins that are produced are a result of isomerization reactions such as iC₄⇄nC₄ which are limited by equilibrium. The C₃ hydrocarbons that are produced are a result of the disproportionation reaction 2iC₄→C₃+iC₅.

TABLE 1

| COMPONENTS (wt %) | FEED A | PRODUCT A |
|---|---|---|
| H₂ | — | 0.2 |
| C₁ | — | 0.1 |
| C₂ | — | 0.1 |
| C₃ | — | 1.3 |
| iC₄ | 0.0 | 9.6 |
| nC₄ | 0.0 | 3.8 |
| iC₅ | 96.8 | 53.4 |
| nC₅ | 3.1 | 18.0 |
| iC₆ | 0.0 | 11.8 |
| nC₆ | 0.0 | 1.7 |
| Cyclopentane (CP) | 0.0 | 0.0 |
| Methylcyclopentane (MCP) | 0.0 | 0.0 |
| Cyclohexane (CH) | 0.0 | 0.0 |
| Benzene (BZ) | 0.0 | 0.0 |
| C₇+ | 0.0 | 0.2 |
| SUM | 100.0 | 100.0 |
| iC₅ converted (%) | — | 44.9 |
| nC₅ + nC₆ | 3.1 | 19.8 |
| C₂, C₃, nC₄, nC₅, nC₆ | 3.1 | 24.9 |

As can be seen in Table 1, the sum of C₂ to C₆ normal paraffins was higher at 24.9 wt % when including the products from disproportionation as compared to 19.8 wt % yield when only including the nC₅+nC₆ hydrocarbons from C₅ and C₆ isomerization reactions.

In a second experiment, another chlorided-alumina catalyst that contained platinum was loaded and operated under isomerization conditions of 3.1 MPa (450 psig), a 0.2 outlet H₂/HC mole ratio and a rate 2 h⁻¹ LHSV with an average temperature at about 176.6° C. (350° F.). Feed B was rich in iC₅ and iC₆ hydrocarbons, contained 1.46% cyclopentane, and trace amounts of C₆ cyclics (0.06 wt % MCP and 0.03 wt % CH). Feed C was similar to Feed B with 1.42 wt % cyclopentane but also contained 1.29 wt % CH (a C₆ cyclic hydrocarbon) and a trace of MCP (0.06 wt %). With the increased amount of C₆ cyclic hydrocarbons in Feed C, as shown in Table 2, the amount of C₃ and C₄ hydrocarbons produced were greatly reduced (compare Product C to Product B). This demonstrates that the increased concentration of the C₆ cyclic hydrocarbons significantly decreased the disproportionation reactions.

TABLE 2

| | FEED | PRODUCT | FEED | PRODUCT |
|---|---|---|---|---|
| COMPONENTS (wt %) | B | B | C | C |
| H₂ | | 0.5 | | 0.5 |
| C₁ | | 0.2 | | 0.1 |
| C₂ | | 0.3 | | 0.1 |
| C₃ | | 3.6 | | 1.0 |
| iC₄ | 0.0 | 9.2 | 0.0 | 3.2 |
| nC₄ | 0.0 | 4.5 | 0.0 | 1.0 |
| iC₅ | 56.1 | 34.1 | 56.1 | 39.8 |
| nC₅ | 1.8 | 11.5 | 1.8 | 13.3 |
| iC₆ | 39.4 | 31.4 | 38.2 | 34.6 |
| nC₆ | 1.1 | 4.6 | 1.1 | 5.1 |
| CP, MCP, CH, BZ | 1.5 | 0.0 | 2.8 | 0.8 |
| C₇+ | 0.0 | 0.2 | 0.0 | 0.7 |
| SUM | 100.0 | 100.0 | 100.0 | 100.0 |
| iC₅ + iC₆ converted (%) | — | 31.4 | — | 21.2 |
| C₂, C₃, nC₄, nC₅, nC₆ | 2.9 | 24.4 | 2.9 | 20.4 |
| CYCLICS | | | | |
| CP | 1.46 | 0.00 | 1.42 | 0.56 |
| MCP | 0.06 | 0.01 | 0.06 | 0.12 |
| CH | 0.03 | 0.01 | 1.29 | 0.08 |
| BZ | 0.00 | 0.00 | 0.00 | 0.00 |
| C₇+ Cyclics | 0.00 | 0.03 | 0.00 | 0.37 |
| TOTAL CYCLICS | 1.54 | 0.05 | 2.77 | 1.12 |
| Cyclics Converted (%) | — | 97.0 | — | 59.5 |
| CP Conversion (%) | — | 100.0 | — | 60.9 |
| nC₄/(nC₄ + iC₄) | — | 32.8 | — | 23.8 |
| nC₅/(nC₅ + iC₅) | 3.1 | 25.2 | 3.1 | 25.1 |
| nC₆/(nC₆ + iC₆) | 2.7 | 12.8 | 2.8 | 12.8 |

As shown in Table 2, the iC₅ hydrocarbons and iC₆ hydrocarbons conversion and the ring opening conversions were lower when the C₆ cyclic hydrocarbons were increased in Feed C. The C₄ isomerization activity was also lower with the increased presence of the C₆ cyclic compounds as demonstrated by the lower nC₄/(nC₄+iC₄) ratio in Product C compared to Product B. Further, the sum of C₂ to C₆ normal paraffins in Product B was 24.4 wt % whereas with the increased C₆ cyclic hydrocarbons in Feed C, the sum of C₂ to C₆ normal paraffins in Product C was less at 20.4 wt %. Lastly, the production of undesired methane was low in Product B.

As discussed above, the extent of disproportionation can be altered by controlling the C₆ cyclic concentration between, for example, 0.0 wt % and 50 wt % in the stream entering the isomerization zone, preferably between 0.0 wt % and 10 wt %.

Furthermore, the ability to adjust the disproportionation reactions, the ring opening reactions and the C₄ isomerization activity will allow for product streams to be generated in the isomerization zone based upon the needs of the downstream processing zones and units. The desirability of such processes is discussed above.

It is also contemplated that iC₄, iC₅ and iC₆ hydrocarbons components can be combined in the hydrotreated naphtha feed and processed together based upon the results of this example. Such a process can eliminate the need for a second downstream isomerization zone for the conversion of iC₄ hydrocarbons to nC₄ hydrocarbons.

To demonstrate that iC₄ hydrocarbons can isomerize in the presence of significant concentrations of C₅, C₆ and C₇+ hydrocarbons in the absence of C₆ cyclic hydrocarbons, a chlorided-alumina catalyst that contained platinum was loaded and operated under isomerization conditions of 3.1 MPa (450 psig), at a rate of 2 h⁻¹ LHSV, with an 0.1 outlet H₂/HC mole ratio, and with an average catalyst bed temperature of 190.5° C. (375° F.). A feed stream (Feed D) was used which was rich in C₅ and C₆ hydrocarbons, trace amount of C₄ components, 0.2 wt % C₇+ hydrocarbon components and no C₆ cyclic hydrocarbons (see Table 3).

TABLE 3

| COMPONENTS (wt %) | FEED D | PRODUCT D |
|---|---|---|
| H₂ | | 0.3 |
| C₁ | | 0.1 |
| C₂ | | 0.2 |
| C₃ | | 4.8 |
| iC₄ | 0.01 | 14.7 |
| nC₄ | 0.01 | 7.1 |
| iC₅ | 55.5 | 29.5 |
| nC₅ | 1.9 | 10.7 |
| iC₆ | 41.9 | 26.9 |
| nC₆ | 0.5 | 4.2 |
| Cyclopentane | 0.0 | 0.1 |
| C₇+ | 0.2 | 1.6 |
| SUM | 100.0 | 100.0 |
| RATIOS (%) | | |
| nC₄/(nC₄ + iC₄) | — | 32.6 |
| nC₅/(nC₅ + iC₅) | 3.2 | 26.6 |
| nC₆/(nC₆ + iC₆) | 1.1 | 13.4 |

While the feed did not contain appreciable concentrations of C₄ hydrocarbons, significant amounts of iC₄ hydrocarbons are produced through disproportionation reactions and nC₄ hydrocarbons are produced through isomerization reactions. Additionally, some lesser amounts of C₄ hydrocarbons may be produced through cracking reactions of heavier components.

Table 3 shows that the C₄ isomerization activity as demonstrated by the nC₄/(nC₄+iC₄) ratio in Product D was similar to the nC₄/(nC₄+iC₄) ratio for Product B even though Product D contained a significantly larger concentration of C₇+ hydrocarbons in the product (1.6 wt % vs. 0.2 wt %). In the absence of C₆ cyclic hydrocarbons, the high C₄ hydrocarbons isomerization activity is achieved in the presence of C₅+ hydrocarbons concentrations of over 40 wt % and specifically in the presence of 1.6 wt % C₇+ hydrocarbons which is significantly higher than the observed limits for other C₄ isomerization processes (the C₄ hydrocarbon isomerization activity has been observed elsewhere to be suppressed by C₅+ hydrocarbon concentrations greater than about 3.0 wt %, and C₆+ hydrocarbon concentrations greater than about 0.1 wt %, and C₇+ hydrocarbon concentrations greater than about 0.001 wt %.).

This example provides evidence that hydrotreated naphtha feed steams can contain iC₄ hydrocarbons which are passed into an isomerization zone in accordance with the present invention may achieve high C₄ isomerization activities to form nC₄ hydrocarbons under isomerization conditions. Thus, it is contemplated that the iC₄, iC₅ and iC₆ hydrocarbons components can be processed together.

Finally, based upon the above, it is also contemplated that similar results are expected with respect to C₇+ cyclic hydrocarbons as those observed for the C₆ cyclic hydrocarbons.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for increasing a conversion of iC₅ hydrocarbons and iC₆ hydrocarbons from a naphtha stream, the process comprising:
isomerizing iC₅ hydrocarbons to normal pentane, under isomerization conditions in the presence of a catalyst, in an isomerization zone;
simultaneously isomerizing iC₆ hydrocarbons to normal hexane, under isomerization conditions in the presence of a catalyst, in the isomerization zone; and,
promoting disproportionation reactions within the isomerization zone by reducing an amount of C₆+ cyclic hydrocarbons entering into the isomerization zone.

2. The process of claim 1 wherein the disproportionation reactions produce at least one of: C₃ hydrocarbons; C₄ hydrocarbons; and, C₇ hydrocarbons.

3. The process of claim 2 wherein at least a portion of the C₄ hydrocarbons, C₇ hydrocarbons, or both are non-normal paraffins.

4. The process of claim 3 further comprising:
isomerizing a portion of the non-normal paraffins portion of the C₄ hydrocarbons and C₇ hydrocarbons to normal paraffins in the isomerization zone.

5. The process of claim 1 further comprising:
promoting a ring opening of C₅ cyclic hydrocarbons in the isomerization zone by reducing an amount of C₆ cyclic hydrocarbons entering into the isomerization zone.

6. The process of claim 1 wherein a conversion of iC₅ hydrocarbons is greater in the isomerization zone with the reduced amount of C₆ cyclic hydrocarbons than a conversion of iC₅ hydrocarbons in the isomerization zone without the amount of C₆ cyclic hydrocarbons being reduced.

7. The process of claim 1 wherein a conversion of iC₆ hydrocarbons is greater in the isomerization zone with the reduced amount of C₆ cyclic hydrocarbons than a conversion of iC₆ hydrocarbons in the isomerization zone without the amount of C₆ cyclic hydrocarbons being reduced.

8. The process of claim 7 wherein a conversion of iC₅ hydrocarbons is greater in the isomerization zone with the reduced amount of C₆ cyclic hydrocarbons than a conversion of iC₅ hydrocarbons in the isomerization zone without the amount of C₆ cyclic hydrocarbons being reduced.

9. A process for producing normal paraffins from at least a portion of a naphtha stream, the process comprising:
isomerizing iC₅ hydrocarbons to normal pentane, under isomerization conditions in the presence of a catalyst, in an isomerization zone;
simultaneously isomerizing iC₆ hydrocarbons to normal hexane, under isomerization conditions in the presence of a catalyst, in the isomerization zone; and,
promoting disproportionation reactions in the isomerization zone to increase an amount of iC₅ hydrocarbons and iC₆ hydrocarbons converted to C₂, C₃, C₄, C₅, C₆ and C₇ normal paraffins.

10. The process of claim 9 wherein promoting disproportionation reactions in the isomerization zone comprises:
reducing an amount of C₆ cyclic hydrocarbons in the isomerization zone.

11. The process of claim 9 further comprising:
promoting a ring opening of C₅ cyclic hydrocarbons in the isomerization zone.

12. The process of claim 9 wherein the disproportionation reactions produce at least one of: C₃ hydrocarbons; iC₄ hydrocarbons; and, iC₇ hydrocarbons.

13. The process of claim 12 further comprising:
isomerizing the produced iC₄ hydrocarbons and iC₇ hydrocarbons to normal paraffins in the isomerization zone.

14. A process for producing normal paraffins from a naphtha stream, the process comprising:
isomerizing iC₅ hydrocarbons to normal pentane, under isomerization conditions in the presence of a catalyst, in an isomerization zone;
simultaneously isomerizing iC₆ hydrocarbons to normal hexane, under isomerization conditions in the presence of a catalyst, in the isomerization zone; and,
increasing a yield of normal paraffins from the isomerization zone by promoting disproportionation reactions and ring opening of C₅ cyclic hydrocarbons within the isomerization zone.

15. The process of claim 14, wherein the yield is increased by reducing an amount of C₆ cyclic hydrocarbons entering into the isomerization zone.

16. The process of claim 14 further comprising:
isomerizing a portion of the iC₄ hydrocarbons and iC₇ hydrocarbons produced by disproportionation reactions in the isomerization zone.

17. The process of claim 1 where the catalyst is selected from the group consisting of: chlorided alumina catalyst; sulfated zirconium catalyst: tungstated zirconia catalyst; and, zeolite-containing catalyst.

18. A process for increasing a conversion of iC₄ hydrocarbons, iC₅ hydrocarbons, and iC₆ hydrocarbons from a naphtha stream, the process comprising:
isomerizing iC₄ hydrocarbons to normal butane, under isomerization conditions in the presence of a catalyst, in an isomerization zone;
isomerizing iC₅ hydrocarbons to normal pentane, under isomerization conditions in the presence of the catalyst, in the isomerization zone;
simultaneously isomerizing iC₆ hydrocarbons to normal hexane, under isomerization conditions in the presence of the catalyst, in the isomerization zone; and,
promoting disproportionation reactions within the isomerization zone by reducing an amount of C₆ cyclic hydrocarbons entering into the isomerization zone.

19. The process of claim 18 further comprising:
increasing a yield of normal paraffins from the isomerization zone by promoting disproportionation reactions and promoting ring opening of C₅ cyclic hydrocarbons within the isomerization zone.

20. The process of claim 19 wherein the ring opening of C₅ cyclic hydrocarbons is promoted by reducing an amount of C₆ cyclic hydrocarbons entering into the isomerization zone.
